(19) United States
(12) Patent Application Publication
(10) Pub. No.: US 2020/0168319 A1
(43) Pub. Date: May 28, 2020

(54) COMPUTATIONAL ARCHITECTURE FOR MANAGING MEDICAL IMAGES

(71) Applicants: Evan Gragg, Mesa, AZ (US); Christopher Bacon, Dix Hills, NY (US); Joshua Bacon, Dix Hills, NY (US); Christopher Bacon, Dix Hills, NY (US)

(72) Inventors: Evan Gragg, Mesa, AZ (US); Christopher Bacon, Dix Hills, NY (US); Joshua Bacon, Dix Hills, NY (US); Christopher Bacon, Dix Hills, NY (US)

(21) Appl. No.: 16/697,440

(22) Filed: Nov. 27, 2019

Related U.S. Application Data
(60) Provisional application No. 62/772,137, filed on Nov. 28, 2018.

(51) Int. Cl. G16H 30/20; G16H 30/40; G06F 21/32; G06T 7/00; G06F 21/33

(52) U.S. Cl. CPC G16H 30/20; G16H 30/40; G06T 2207/10072; G06T 7/0012; G06F 21/33; G06F 21/32

(57) ABSTRACT

A computational architecture for managing medical images includes a first server. The architecture includes a medical imaging device, wherein the medical imaging device receives instructions from the first server. A first image being taken by the medical imaging device is in a first format. The first server is communicable with a primary database through a firewall, the first server deposits the first image into a primary database. A second server is communicable with the primary database, wherein the second server transforms the first image into a second image. The second image is in a second format different from the first format.

### PRIORITY

This application claims the benefit of priority of U.S. Patent Application Ser. No. 62/772,137, filed on Nov. 28, 2019, which is hereby incorporated by reference herein.

### FIELD OF THE DISCLOSURE

The present invention relates generally to methods and systems for managing medical images. More particularly, the present invention relates to computational architecture for managing medical images, wherein, compared to traditional solutions, the architecture is more efficient and more secure in managing and distributing medical images. Further, the systems and methods disclosed based on the computational architecture herein have improved interoperability compared to current solutions. In fact, the embodiments disclosed herein provide medical images compatible with any commercially available image browsers as well as electronic medical records (EMR).

### BACKGROUND

Medical images, e.g., X-ray, CT, MRI, ultrasound, etc., are widely used in the practice of modern medicines. Almost all medical images are formatted in Digital Imaging and Communications in Medicine (DICOM). Currently, DICOM defines the formats for medical images that can be exchanged with the data and quality necessary for clinical use.

DICOM compatible EMR systems are very expensive and is not practical for smaller facilities. Some facilities that have DICOM compatible EMR systems want to limit storage because DICOM files are large and the cloud storages are charged for the amount of storage being used. Laws require storage for 5 to 6 years of medical images. However, DICOM is incompatible with any other commercially available image formats. Thus, medical images in DICOM cannot be viewed with cell phone, desktop, and/or laptop that do not have DICOM compatible browsers. The incompatibility limits the sharing of the medical images, which further limits the flexibility of the practice of medicine. Further, DICOM is an image format that consumes a lot of computation resources and memory, at least partially, because the file size of DICOM is large. Thus, just downloading a DICOM image requires a lot of computational power. If a user of the image browsing device wants to manipulate the images, e.g., zoom-in, zoom-out, adjusting contrast, adjusting brightness, regarding a particular portion of the DICOM image, enormous amount of computational resources are required, slowing down or even crashing the entire system for processing other tasks.

The embodiments disclosed herein are directed to a computational architecture for managing medical images. Compared to traditional solutions, the computational architecture disclosed herein is more efficient and more secure in managing and distributing medical images. Further, the systems and methods built based on the architecture are compatible with any commercially available image browsers. The embodiments disclosed herein allow users of image browsing devices to manipulate the images on any image viewing device, e.g., cell phone, desktop, laptop, and/or tablet, efficiently without compromising the performance of other tasks simultaneously running on the device. This is because the embodiments disclosed herein can utilize image formats that are much smaller than DICOM without compromising the quality of the images, especially the area of interest of the images.

### SUMMARY

The present invention relates generally to methods and systems for managing medical images. More particularly, the present invention relates to computational architecture for managing medical images, wherein, compared to traditional solutions, the architecture is more efficient and more secure in managing and distributing medical images. Further, the systems and methods disclosed based on the computational architecture herein have improved interoperability compared to current solutions. In fact, the embodiments disclosed herein provide medical images compatible with any commercially available image browsers.

In one embodiment, the computational architecture is more efficient compared to current solution because the architecture is able to transform DICOM format to commonly available commercial image formats, such as JPG, GIF, PNG, TIFF, BMP, or the like without limitation. Such commercial image formats require much less memory resource to store and much less computational recourse to process compared to DICOM format. The improved efficiency is gained from using commercial image formats, such as JPG, GIF, PNG, TIFF, BMP, or the like because smaller file size translates to less computational resources being required, quicker processing speed, and faster device-to-device data transfer.

The embodiments of the computational architectures and methods thereof do not compromise the quality of the image. This is achieved because the transformations to the new image in commercial formats, such as JPG, GIF, PNG, TIFF, BMP, or the like, are encoded to have 0% image loss during compression. Therefore, the newly created image is much smaller in size, yet pertains sufficient quality of the image for practicing medicine and/or diagnostics.

DICOM is not a standard image format that regular image browser can operate with. However, commercially available image formats, such as JPG, GIF, PNG, TIFF, BMP, or the like are universally operable with almost on any image or website browsing software, the computational architecture disclosed herein is compatible with any cell phones, desktops, laptops, and tablets currently available. Thus, by providing the ability to transform DICOM to JPG, GIF, PNG, TIFF, BMP, or the like, the computational architecture disclosed herein provides improved interoperability for any computation system or mobile device. For example, a medical doctor in a remote area can easily download and view the medical image in JPG format, with the area-of-interest sufficiently represented, using a regular mobile phone (Android or Apple OS, regardless).

The computational architecture has improved security because it includes a double backup system. A primary database is configured to store DICOM parsed data including recreating the DICOM image (DICOM viewer). A secondary database stores the converted DICOM images from primary database only. In situations that the primary database is compromised, the secondary database already has the converted images from the primary database. However, the primary database has no communication with the secondary database.

In one embodiment, a computational architecture for managing medical images includes a first server. A medical imaging device receives the Worklist from the first server. A first image being taken by the medical imaging device is in a first format, wherein the first server is communicable with a primary database through a firewall, and the first server deposits the first image into a primary database. A second server is communicable with the primary database, wherein the second server transforms the first image into a second image and the second image is in a second format different from the first format. In one embodiment, the first server is the same as the second server. This means a single server serves both as the medical imaging device control server as well as the client serving server.

In one embodiment, a computing architecture for managing medical images includes a server. The server includes at least a processor and a machine readable memory accessible by the processor. The server performs the followings: receiving a request from an image browsing device to access a first image, the first image being in a first format; conducting an authentication process for the image browsing device; conducting an authorization process for the image browsing device, accessing the first image from a primary database; transforming the first image into a second image, the second image being in a format different from the first format; and sending the second image to the image browsing device.

The foregoing has outlined rather broadly the features and technical advantages of the present invention such that the detailed description of the invention that follows may be better understood. Additional features and advantages of the invention will be described hereinafter that form the subject of the claims of the invention. It should be appreciated by those skilled in the art that the concepts and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the disclosed systems and methods, reference is now made to the following descriptions taken in conjunction with the accompanying drawings.

FIG. 1 is a schematic diagram of a computational architecture for managing medical images according to one embodiment of the disclosure.

FIG. 2 is a method for managing medical images according to one embodiment of the disclosure.

FIG. 3 is a schematic diagram of information flow of the computational architecture according to one embodiment of the disclosure.

FIG. 4 is a flow chart of a computational architecture for managing medical images according to one embodiment of the disclosure.

FIG. 5 is schematic diagram of a computational architecture for managing medical images according to one embodiment of the disclosure.

FIG. 6 is a schematic diagram of information flow of the computational architecture according to one embodiment of the disclosure.

FIG. 7 is a graphical user interface (GUI) framework according to one embodiment of the disclosure.

FIG. 8 is a graphical user interface (GUI) framework according to one embodiment of the disclosure.

FIG. 9 is a graphical user interface (GUI) framework according to one embodiment of the disclosure.

FIG. 10 is a graphical user interface (GUI) framework according to one embodiment of the disclosure.

FIG. 11 is a graphical user interface (GUI) framework according to one embodiment of the disclosure.

FIG. 12 is a graphical user interface (GUI) framework according to one embodiment of the disclosure.

FIG. 13 is a graphical user interface (GUI) framework according to one embodiment of the disclosure.

FIG. 14 is a graphical user interface (GUI) framework according to one embodiment of the disclosure.

### DETAILED DESCRIPTION

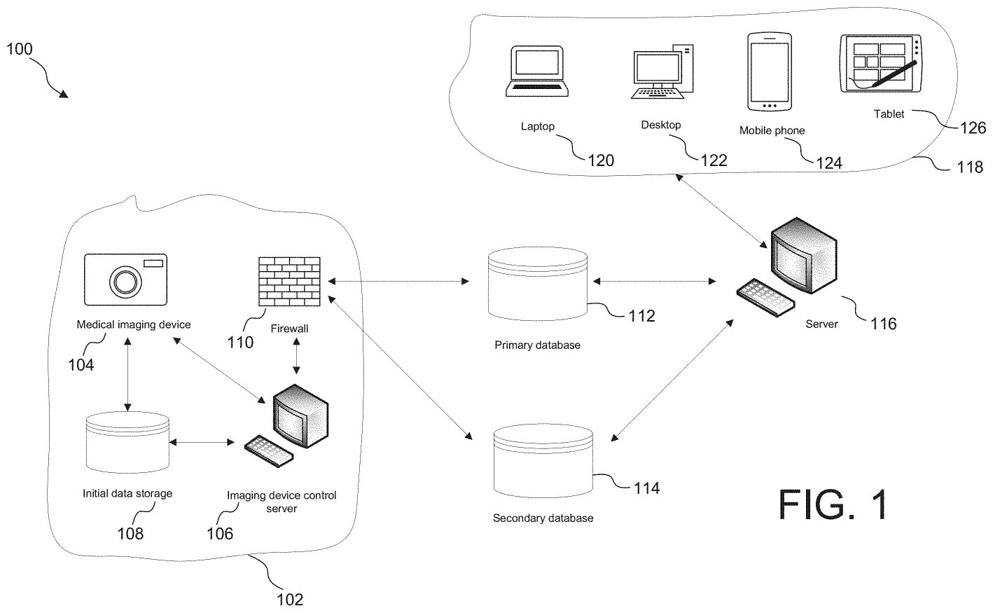

FIG. 1 is a schematic diagram of a computational architecture 100 for managing medical images according to one embodiment of the disclosure. The computational architecture 100 includes a clinic 102. The clinic 102 can be a medical doctor office, a hospital, a wellness center, an assisted living center, or the like.

The clinic 102 includes a medical imaging device 104. The medical imaging device 104 can be X-ray, CT, MRI, ultrasound, etc. In one example, the medical imaging device 104 can be used for diagnosing bone conditions, e.g., crack, fracture, etc. In another example, the medical imaging device 104 can be used for diagnosing soft tissue conditions, e.g., pregnancy, tumor, tissue inflammation, etc.

The medical imaging device 104 is controlled by an imaging device control server 106. The server 106 communicates with the medical imaging device 104. The server 106 provides instructions for the imaging device 104 to take one or more medical images over a certain period of time with variable sampling rates.

The medical imaging device 104 takes the image in DICOM format (hereinafter medical images in DICOM format are referred as “DICOM image”). The DICOM image taken by the imaging device 104 is sent to an initial database 108. The initial database 108 communicates with the server 106. The server 106 is compatible with DICOM image. In one example, the image browser of the server 106 is picture archiving and communication system (PACS).

The operator of the server 106, e.g., a technician, a nurse, a medical doctor, etc., may review the DICOM image and add labels and/or comments to the image. When the image was taken by the medical imaging device, information and tags may be automatically generated in association with the image. Such labels, comments, information and tags may include, patient name, study date, date the image created, study description, series description, access number, sex, body part the image was taken for, exposure, performing physician, patient ID, date of birth of the patient, insurance number, radiation time, view position description, study ID, series ID, and instance image number.

Real scale measurements can be added to the DICOM image at the time the image is viewed on the Server. 104. Real scale measurement may be labeled at any place on the image, it can be on the side, top, bottom, or proximal to any of the area-of-interest. The measurement is a mapping of distances with the pixels of the images, e.g., 500, 1000, or 2000 pixels per inch. When the DICOM image is scaled, e.g., zoom-in, zoom-out, the label of the measurement is proportionally scaled.

The imaging device control server 106 accesses the image from the initial data storage 108. The imaging device control server 106 sends the image to the primary database 112 and secondary database 114 through the firewall 110. The firewall 110 may perform encryption functionality to the images, wherein the decryption may not be performed until the client 118 requested the image with appropriate authentication, authorization, and showing appropriate community-of-interest (“COI”) key and/or COI certificate.

In one embodiment, the server 106 or the firewall 110 may encrypt the images with a first key that is associated with the clinic 102. The first key may be a private key associated to the clinic 102. The image may be decrypted with a second key that is associated to a particular community-of-interest. The community-of-interest may be another clinic, hospital, or a department within a hospital. The second key may be a public key associated to a client 118.

It is noted that none of the components of the clinic 102 is directly exposed to the internet, except the firewall 110. The encrypted image may be sent through the firewall 110 to be deposited with both the primary database 112 and secondary database 114.

The server 116 is in communication with the primary database 112 and the secondary database 114. The client 118 is connected to the server 116. The client 118 may be a doctor, a clinic, a patient, an assisted living facility, etc. In one embodiment, the client 118 may be a different department of a same hospital of the clinic 102. In another embodiment, the client 118 may be the same, or the same entity, of the clinic 102. The client 118 includes various image browsing devices, e.g., a laptop 120, a desktop 122, a mobile phone 124, a tablet 126, etc. Each of the browsing devices are equipped with standard picture/image browsing software, e.g., Goggle Chrome, Microsoft IE, Adobe, etc.

When the request for an image is received by the server 116 from the client device 120, 122, 124, 126, the server 116 performs the authentication process by checking the username, the password, finger prints, user voice, and/or other biometric data. The server 116 may also perform the authorization process. In one embodiment, the authorization process includes checking whether the client device 120, 122, 124, 126 presents the right community-of-interest key that can be used to decrypt the image stored in the primary database 112.

If the client 118 is authenticated and authorized, the server 116 retrieves the requested image from the database 112. The server 116 decrypts the image with the decryption key provided by the client 118. In one embodiment, at this point, the decrypted image is DICOM image as originally taken by the medical imaging device 104. In one embodiment, the server 116 may send the client 118 the DICOM image.

The server 116 may transform the DICOM image to other commercially available image formats, such as JPG, GIF, PNG, TIFF, BMP, or the like. The server 116 may perform a zoom-in on the DICOM image. The server 116 may zoom-in at an area-of-interest on the DICOM image, up to 36×, without limitation, and take a snapshot of the area-of-interest and transform the area-of-interest to JPG, GIF, PNG, TIFF, and BMP, or the like (hereinafter “non-DICOM” format). The transformed non-DICOM image includes all the labels, comments, information and tags may include, patient name, study date, date the image created, study description, series description, access number, sex, body part the image was taken for, exposure, performing physician, patient ID, date of birth of the patient, insurance number, radiation time, view position description, study ID, series ID, and instance image number. The transformed non-DICOM image also includes the real scale measurement.

In one embodiment, the user of the client device 120, 122, 124, 126 may perform a series of zoom-in, zoom-out, adjusting brightness, adjusting contrast, or the like on the image. In response, the server 116 takes a series of snapshots of the zoomed-in or zoomed-out images in non-DICOM format and send it to the client device 120, 122, 124, 126.

Compared to traditional solutions, the computational architecture 100 is more efficient and more secure in managing and distributing medical images. Further, the systems and methods disclosed based on the computational architecture 100 herein have improved interoperability compared to current solutions. In fact, the embodiments disclosed herein provide medical images compatible with any commercially available image browsers.

In one embodiment, the computational architecture 100 is more efficient compared to current solution because the architecture is able to transform DICOM format to commonly available commercial image formats, such as JPG, GIF, PNG, TIFF, BMP, or the like without limitation. Such commercial image formats require much less memory resource to store and much less processing power compared to DICOM format at the client's end 118. The improved efficiency is gained from using commercial image formats, such as JPG, GIF, PNG, TIFF, BMP, or the like because smaller file size translates to less computational resources being required, quicker processing speed, and faster device-to-device data transfer.

The embodiments of the computational architectures and methods thereof do not compromise the quality of the image. This is achieved because the computational architecture disclosed herein provides the functionality to zoom-in up to 36 times (36×) of the image in DICOM format and taking a snapshot of the area-of-interest (i.e., zoomed-in area of the DICOM format image) and transform only the area-of-interest to a new image in commercial format, such as JPG, GIF, PNG, TIFF, BMP, or the like. Therefore, the newly created image is in a much smaller file size, yet pertains sufficient quality of the image for practicing medicine, especially the area-of-interest.

DICOM is not a standard image format that is compatible with regular image browsers. On the other hand, commercially available image formats, such as JPG, GIF, PNG, TIFF, BMP, or the like are universally operable with any image or website browsing software. The computational architecture disclosed herein is compatible with any cell phones, desktops, laptops, and tablets currently available. Thus, by providing the ability to transform DICOM to JPG, GIF, PNG, TIFF, BMP, or the like, the computational architecture disclosed herein provides improved interoperability for any computation system or mobile device. For example, a medical doctor in a remote area can easily download a medical image in JPG format, with the area-of-interest sufficiently zoomed-in, using a regular mobile phone (Android or Apple OS, regardless) and view the medical image.

The computational architecture has improved security because it includes a double backup system, e.g., primary database 112 and secondary database 114. A primary database 112 is configured to store the medical images. A secondary database 114 stores the same information as the primary database. In situations that the primary database 112 is compromised, the secondary database 114 replaces the functionality of the primary database. However, the primary database 112 has no communication with the secondary database 114, this is to prevent the cybersecurity risk from affecting the secondary database.

The functionality of the server 116 can be implemented in firmware and/or software. If implemented in firmware and/or software, the functions described above may be stored as one or more instructions or code on a computer-readable medium. Examples include non-volatile computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and blu-ray discs. Generally, disks reproduce data magnetically, and discs reproduce data optically. Combinations of the above should also be included within the scope of computer-readable media.

In addition to storage on computer-readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

The functionality of the server 116 can be implemented as a virtual machine with emulated operating system that runs at client's end 118, such as on the devices 120, 122, 124, 126.

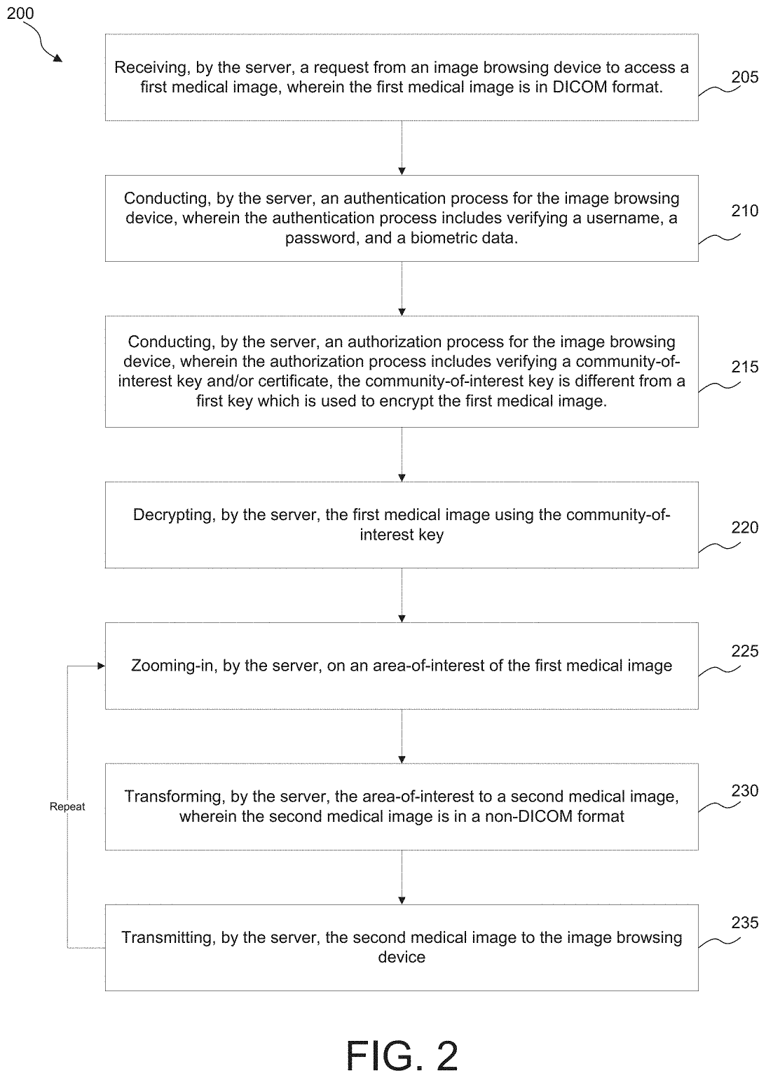

FIG. 2 is a method 200 for managing medical images according to one embodiment of the disclosure. Method 200 can be implemented by the computational architecture 100, wherein similar terms refer to similar elements. The method 200 includes step 205 receiving, by the server 116, a request from an image browsing device to access a first medical image, wherein the first medical image is in DICOM format.

The method 200 includes step 210 conducting, by the server 116, an authentication process for the image browsing device, wherein the authentication process includes verifying a username, a password, and a biometric data.

The method 200 includes step 215 conducting, by the server 116, an authorization process for the image browsing device, wherein the authorization process includes verifying a community-of-interest key, the community-of-interest key is different from a first key which is used to encrypt the first medical image.

The method 200 includes step 220, decrypting, by the server 116, the first medical image using the community-of-interest key.

The method 200 includes step 225, zooming-in, by the server 116, on an area-of-interest of the first medical image. In one example, the area-of-interest can be an area of tumor growth, an area of bone crack, an area of disk displacement, an area of tissue inflammation, etc. Step 225 may include other adjustments of the first medical image, e.g., brightness, contrast, or the like.

The method 200 includes step 230, transforming, by the server 116, the area-of-interest to a second medical image, wherein the second medical image is in a non-DICOM format. The non-DICOM format refers to commonly available commercial image formats, such as JPG, GIF, PNG, TIFF, BMP, or the like without limitation.

The method includes step 235, transmitting, by the server 116, the second medical image to the image browsing device.

As the user of the image browsing device can change the area-of-interest by zooming out, the method 200 may include a step (not shown) of zooming-out, by the server 116, from an area-of-interest of the first medical image. In one example, the area-of-interest can be an area of tumor growth, an area of bone crack, an area of disk displacement, an area of tissue inflammation, etc.

As the user of the image browsing device can change the area-of-interest by shifting the focus to other areas of the first medical image, the method may repeat the zooming in (step 225), zooming out process (above mentioned), transforming the area-of-interest to non-DICOM format (step 230), and transmitting the second medical image to the browsing device (step 235).

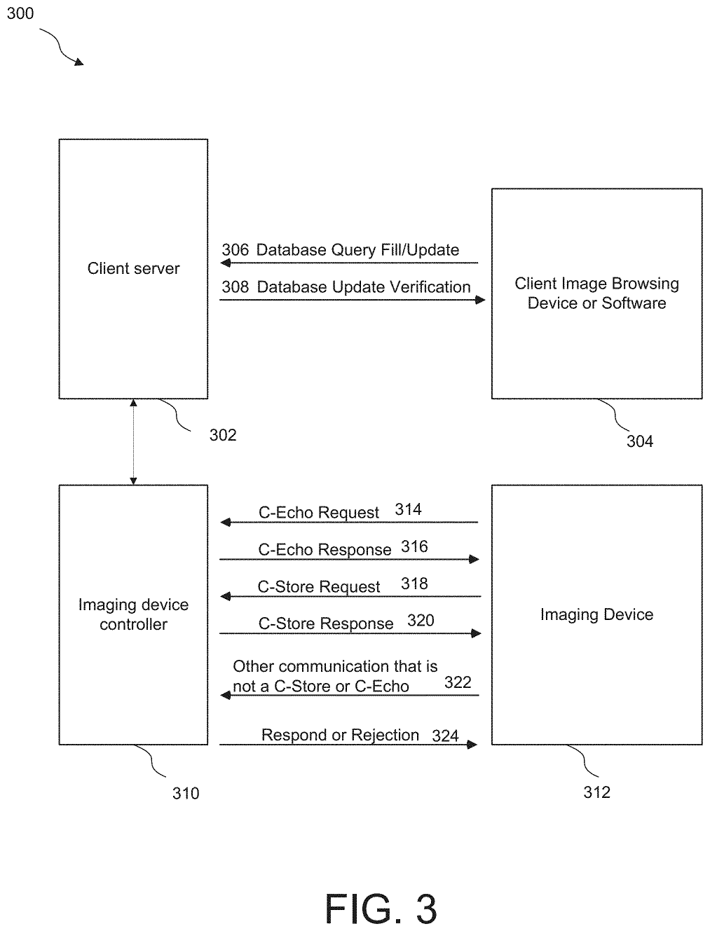

FIG. 3 is a schematic chart of information flow 300 of the computational architecture 100 according to one embodiment of the disclosure.

The imaging device 312 sends a c-echo request 314 (i.e., a request to verify server is online) to the imaging device controller 310. The imaging device 310 responds to the imaging device 316. The imaging device 312 sends the imaging device controller 310 a c-store request 318 (i.e., a request to store an image). The imaging device 312 responds to a c-store response. The imaging device 312 sends the imaging controller 310 other communications that are not c-store or c-echo requests 322. The imaging device controller 310 either responds or rejects the non-c-store or non-c-echo requests.

The imaging device controller 310 communicates with the client server 302. The client image browsing device or software 304 sends the client server 302 a database query fill/update. The client server 302 sends the client image browsing device or software 304 the database update verification.

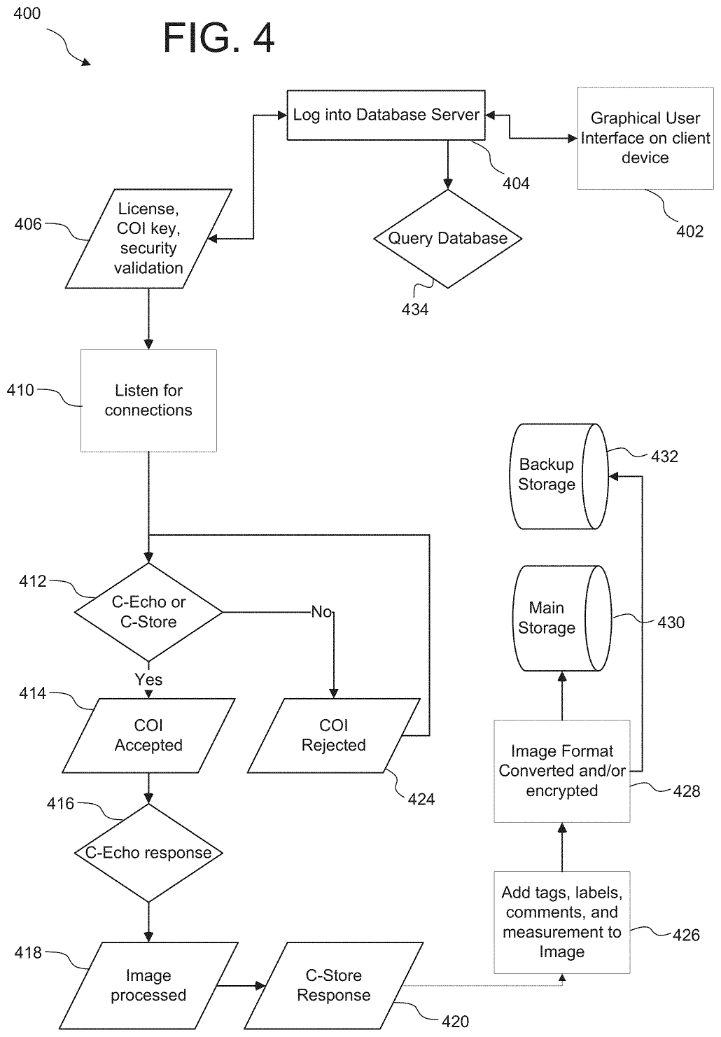

In one embodiment, the client server 302 and imaging device controller 310 can be the same server, facing both the client device 304 as well as the imaging device 312, similar to embodiments shown in FIG. 4.

FIG. 4 is a flow chart of a computational architecture 400 for managing medical images according to one embodiment of the disclosure. In the computational architecture 400, the server 404 serves both the client 304 as well as the imaging device 312.

In the computational architecture 400, the graphical user interface (GUI) on a client device 402 can login to the database server 404. The login process includes authentication, e.g., checking username, password, and biometric data, etc. The GUI 402 can be an application on the client device 118 (a laptop 120, a desktop 122, a mobile phone 124, a tablet 126).

The login from the GUI 402 may include request for medical images. The server 404 may access the query database 434, which logs all activities performed by the GUI 402. The time, identity, and requested query are recorded in the query database 434. Whether the specific GUI 402 was granted or denied of access to the server 404 is also recorded at the query database 434.

The server 404 further performs the authorization at 406. At 406, license, community-of-interest key, community-of-interest certificate, and other security validations are performed. At 410, the server 404 listens for connections from the GUI 402.

In one embodiment, the GUI 402 listens for a request from the medical imaging device to store an image. In such, the GUI 402 may be in similar roles as being both the imaging device control server 106 as well as the client 118. At 412, the server receives a c-echo request. A c-echo request is a request to verify Server is Online. A c-store request is a request to store an image.

At 412, the server may verify the COI key and/or certificate and determine whether the requested action is granted or rejected. The decision of grant or rejection is made, at least partially, based on the verification of COI key and/or certification. Note, COI key is the key shared within the COI that can be used for decrypting the previously encrypted medical images. COI certificate is a certification representing the COI. In some embodiments, COI key and COI certificate can be the same. In some embodiments, COI key and COI certificate can be different.

At 414, the server accepted the COI key/certificate and granted the request. At 424, the server rejected the COI key/certificate and rejected the request. At 416, the server proceeds with the action of c-echo verifying Server is online. At 418, the image is taken using the medical imaging device. At 420, the server grants the c-store request, meaning the image is stored.

At 426, in one embodiment, the image taken by the medical imaging device is in DICOM format. At 426, various tags, labels, real scale measurement, and comments are added to the image.

At 428, the image format may be converted to non-DICOM format and/or encrypted. The image is sent to both the main storage 430 and the backup storage 432.

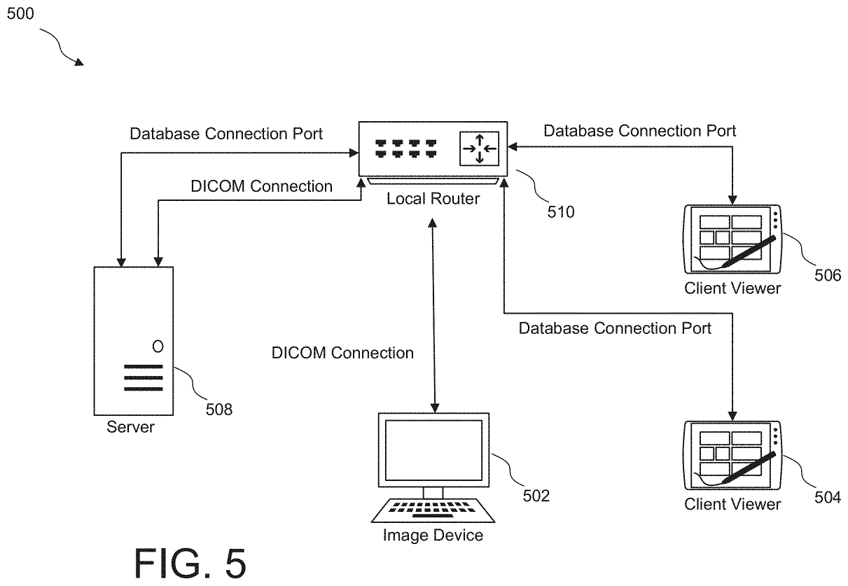

FIG. 5 is schematic diagram of a computational architecture 500 for managing medical images according to one embodiment of the disclosure. The medical imaging device 502 can be X-ray, CT, MRI, ultrasound, etc. The medical image taken by the image device 502 is in DICOM format. The medical image passes through a local router 510 and is stored at server 508.

The server 508 receives requests from the images stored from client viewer 506, 504. The server may interact with the client viewer as described in FIG. 2. The server is capable of transforming the DICOM image to non-DICOM image and send the images requested to the client viewers 504, 506.

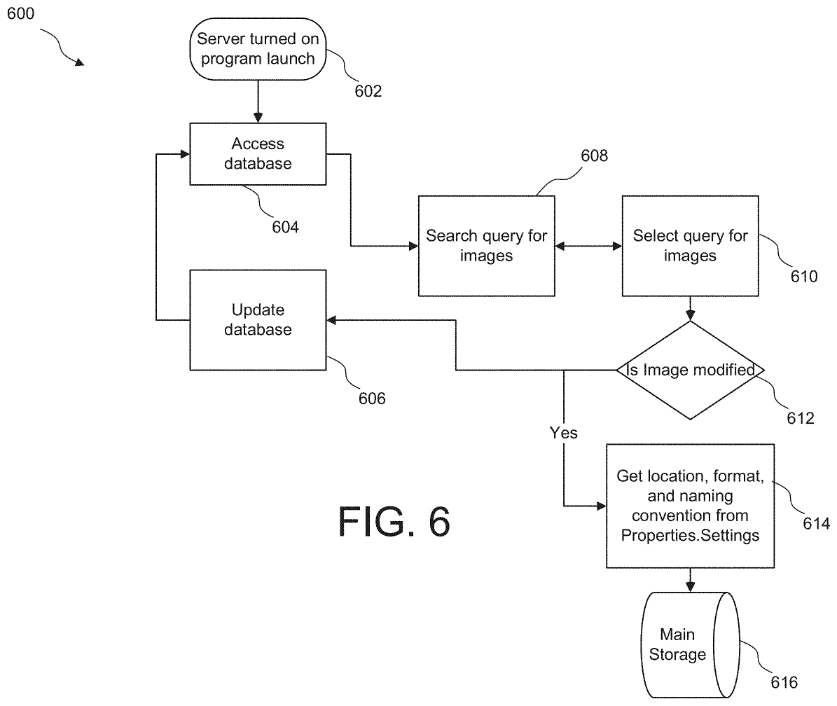

FIG. 6 is a schematic diagram of a computational architecture 600 according to one embodiment of the disclosure.

In the computational architecture 600, the server is turned on at 602. The server 602 can be the imaging device control server 106. The server 602 can also be the client serving server 116. The login process includes authentication, e.g., checking username, password, and biometric data, etc. At 604, the database is accessed.

At 608, the server allows the client to search for a particular image. Various filters are provided for the search function. The filters may include patient name, study date, date the image created, study description, series description, access number, sex, body part the image was taken for, exposure, performing physician, patient ID, date of birth of the patient, insurance number, radiation time, view position description, study ID, series ID, and instance image number.

Based on the search, the database provides a list of images that match with the search query. At 610, the client select an image. The computational architecture 600 allows the client to add comments to the image, e.g., diagnostic notes, etc. At 612, the server checks whether the image is modified. If image is modified, the query database is updated at 606.

The server may get further information for the image at the 614. In one embodiment, the image may lack some of the tags, labels, and comments. The server will examine the tags, labels, and comments of the image and automatically fill in the missing information at 614. In one example, the image is newly created by the imaging device and has no tags, no label, and no comments. The server at 614 may automatically load some of the missing information automatically, e.g., location, format, naming convention, etc. Such information may be defined in a batch program, e.g., “properties.settings.” The server then store the modified images to a main storage 616.

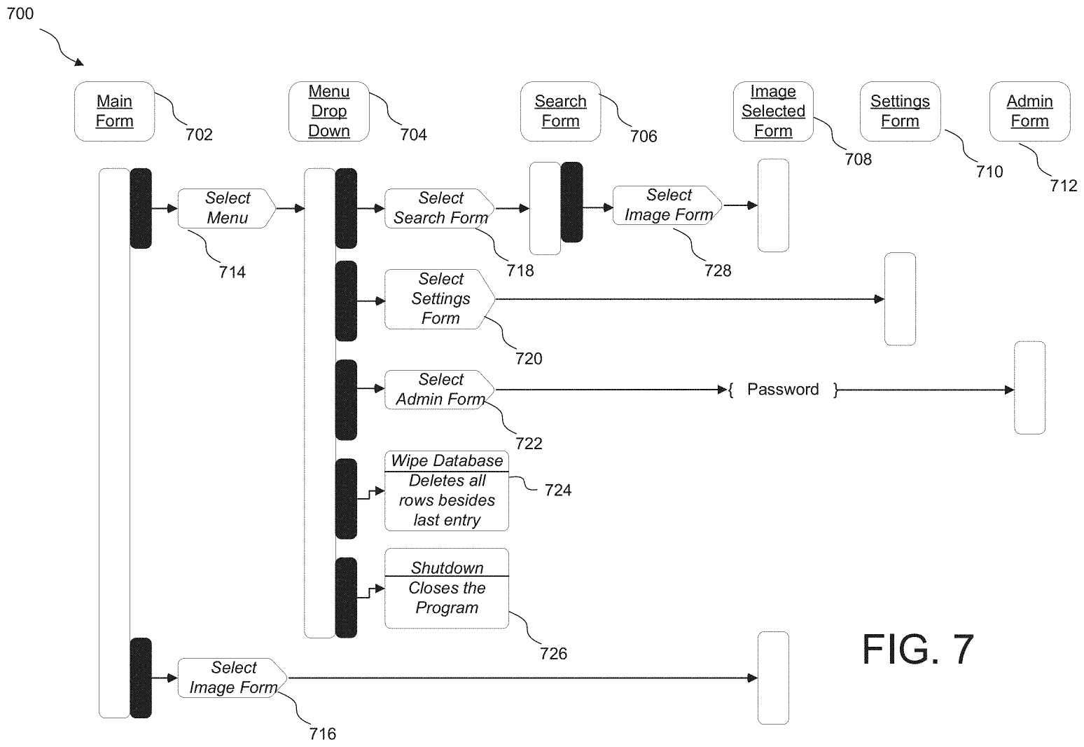

FIG. 7 is a graphical user interface (GUI) framework 700 according to one embodiment of the disclosure. The framework 700 starts from the main form 702 layer. The main form 702 includes select menu 714 and select image form 716.

The second layer is the menu drop down 704. The menu drop down 704 includes select search form 718, select settings form 720, select admin form 722, wipe database 724, shutdown 726.

The third layer is the search form 706. The search form 706 includes select image form 728. The forth layer is the image selected form 708. The fifth layer is settings form 710. The sixth layer is admin form 712.

When activated, the select menu 714 leads the user to the second layer, the menu drop down 704. When activated, the select image form 716 leads the user the fourth layer, the image selected form 708.

When activated, the select search form 718 leads to the select image form 728. When activated, the select settings form 720 leads to the settings form. When activated, the select admin form 722 leads to admin form 712, wherein password may be required to proceed. When activated, wipe database 724 deletes all rows besides last entry. When activated, shutdown 726 closes the program. When activated, the select image form 728 leads to the image selected form 708.

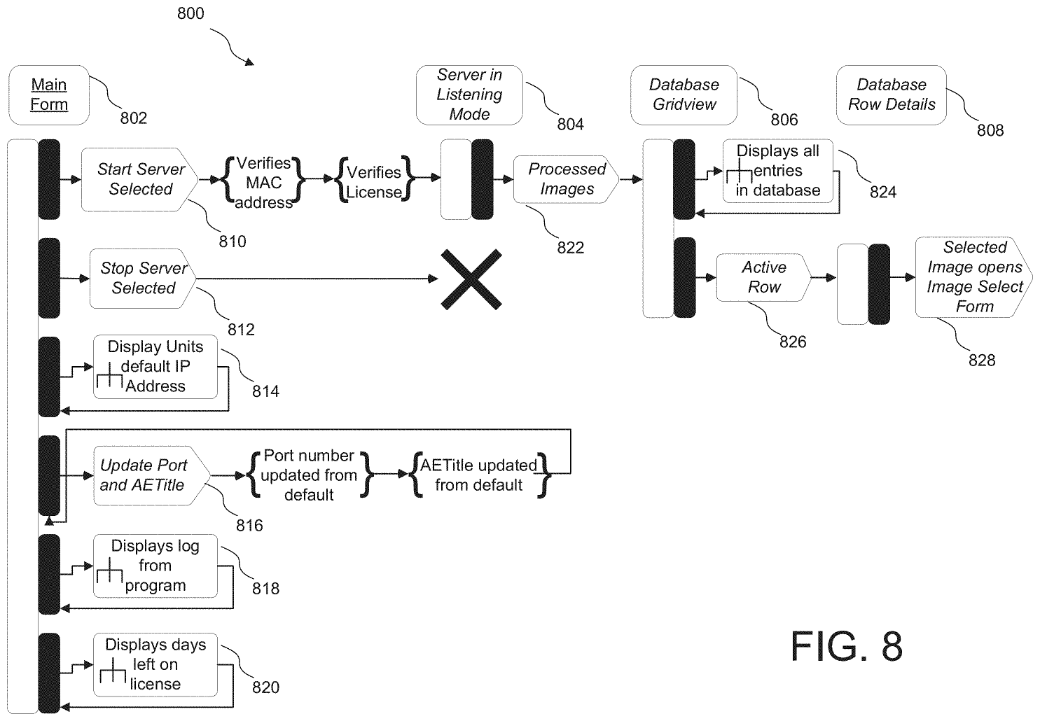

FIG. 8 is a graphical user interface (GUI) framework 800 according to one embodiment of the disclosure. The first layer is main form 802. The second layer is server in listening mode 804. The third layer is database gridview 806. The fourth layer is database row details 808.

The main form 802 includes start server selected 810, stop server selected 812, display units default IP address 814, update port and AE title 816, displays log from program 818, and displays days left on license 820.

When activated, the start server selected 810 leads to processed images 822, wherein MAC address may be verified and the license may be verified. When activated, stop server selected 812 stops the server.

When activated, display units default IP address 814 shows the display units' IP address. When activated, 816 updates port and AE title. When activated, 818 displays log from program. When activated, 820 displays the days left on license. When activated, processed images 822 leads to display all entries in database 824 or active row 823. The active row 826 leads to selected image opens image select form 828.

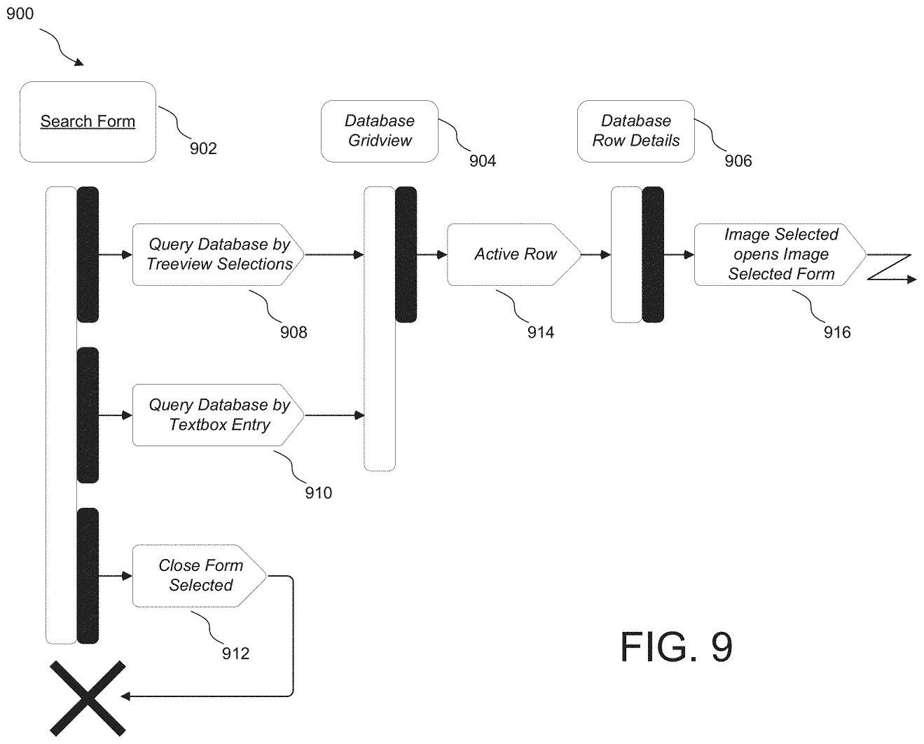

FIG. 9 is a graphical user interface (GUI) framework 900 according to one embodiment of the disclosure. The first layer is search form 902. The second layer is database gridview 904. The third layer is database row details 906.

The search form 902 includes query database by treeview selections 908, query database by textbox entry 910, and close form selected 912.

When activated, query database by treeview selections 908 leads to database gridview 904. When activated, database gridview 904 leads to active row 914. When activated, active row 914 leads to database row details 906.

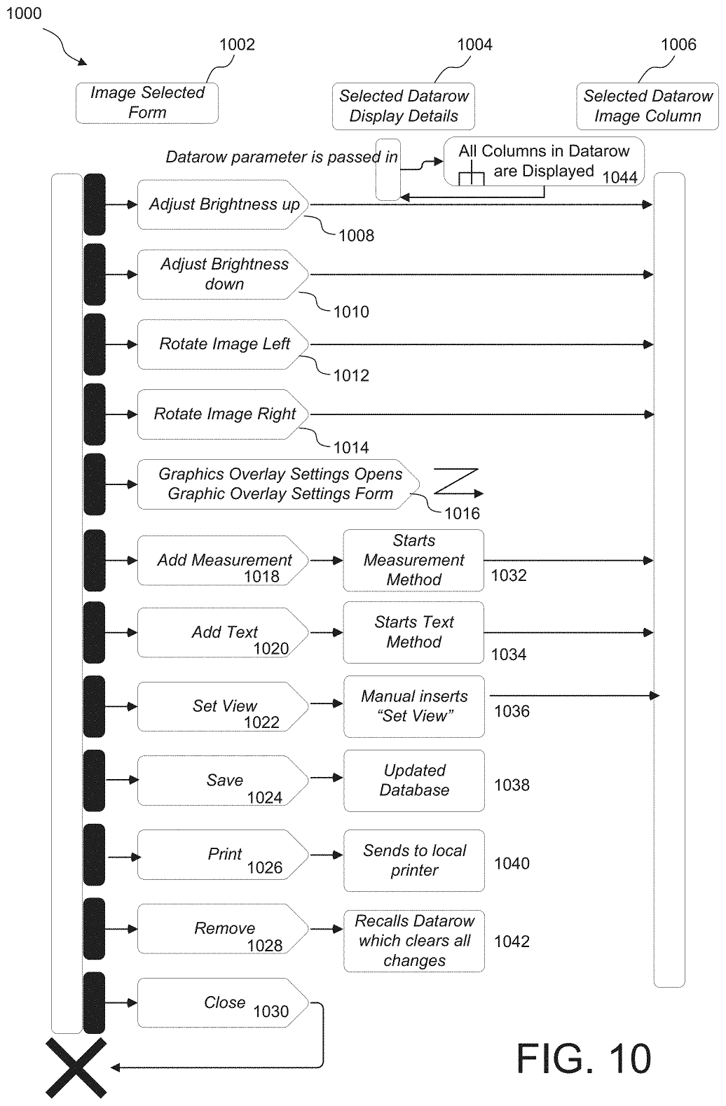

FIG. 10 is a graphical user interface (GUI) framework 1000 according to one embodiment of the disclosure. The first layer is image selected form 1002. The second layer is selected datarow display details 1004. The third layer is selected datarow image column 1006.

The image selected form 1002 includes adjust brightness up 1008, adjust brightness down 1010, rotate image left 1012, rotate image right 1014, graphics overlay settings 1016, add measurement 1018, add text 1020, set view 1022, save 1024, print 1026, remove 1028, close 1030. The selected datarow display details 1004 includes all columns in datarow being displayed 1044.

When activated, add measurement 1018 leads to starts measurement method 1032. When activated, add text 1020 leads to starts text method 1034. When activated, set view 1022 leads to manual inserts “set view” 1036. When activated, save 1024 leads to updated database 1038. When activated, print 1026 leads to sends to local printer 1040. When activated, remove 1028 leads to recalls datarow which clears all changes 1042.

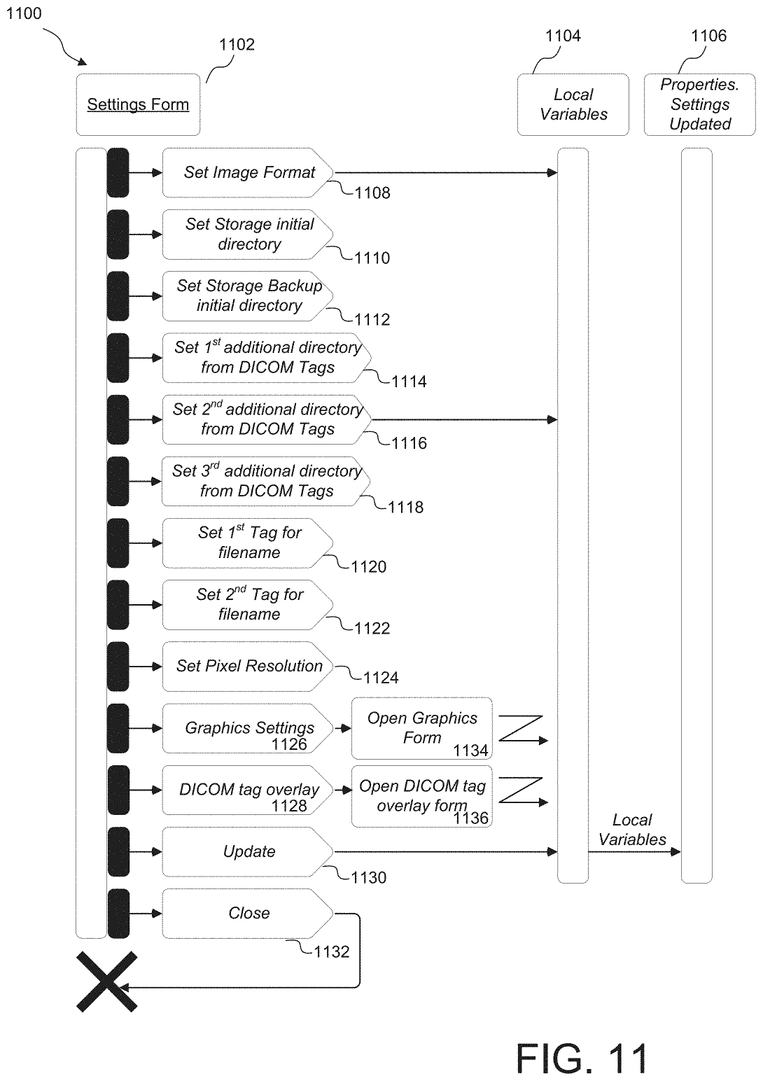

FIG. 11 is a graphical user interface (GUI) framework 1100 according to one embodiment of the disclosure. The first layer is settings form 1102. The second layer is local variables 1104. The third layer is properties (settings updated) 1106.

The settings form 1102 includes set image format 1108, set storage initial directory 1110, set storage backup initial directory 1112, set first additional directory from DICOM tags 1114, set second additional directory from DICOM tags 1116, set third additional directory from DICOM tags 1118, set first tag for filename 1120, set second tag for filename 1122, set pixel resolution 1124, graphics settings 1126, DICOM tag overlay 1128, update 1130, and close 1132.

When activated, graphics settings 1126 leads to open graphics form 1134. When activated, DICOM tag overlay 1128 leads to open DICOM tag overlay form 1136.

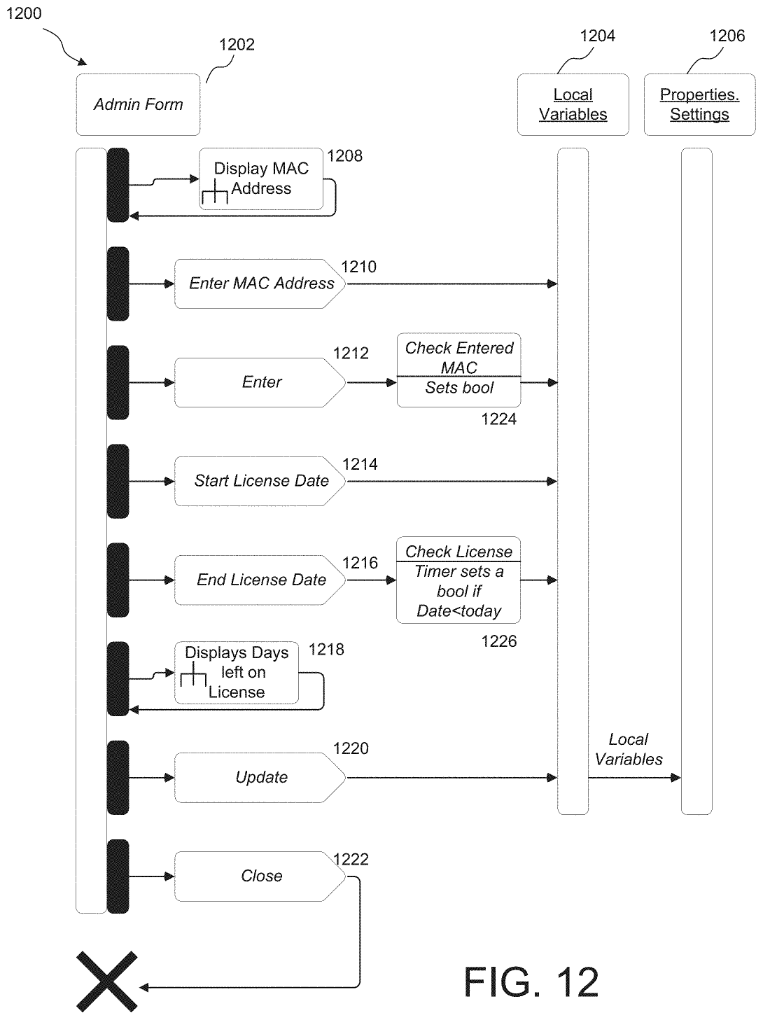

FIG. 12 is a graphical user interface (GUI) framework 1200 according to one embodiment of the disclosure. The first layer is admin form 1202. The second layer is local variables 1204. The third layer is properties settings 1206.

The admin form 1202 includes display MAC address 1208, enter MAC address 1210, enter 1212, start license date 1214, end license date 1216, displays days left on license 1218, update 1220, and close 1222. When activated, enter 1212 leads to check entered MAC 1224. When activated, end license date 1216 leads to check license 1226.

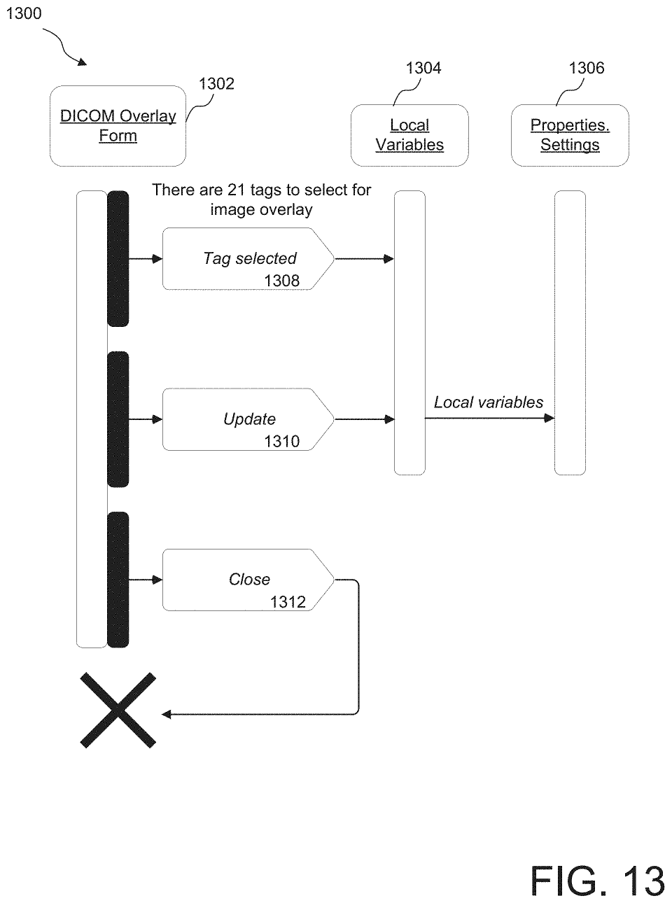

FIG. 13 is a graphical user interface (GUI) framework 1300 according to one embodiment of the disclosure. The first layer is DICOM overlay form 1302. The second layer is local variables 1304. The third layer is properties 1306. The DICOM overlay form 1302 includes tag selected 1308, update 1310, and close 1312.

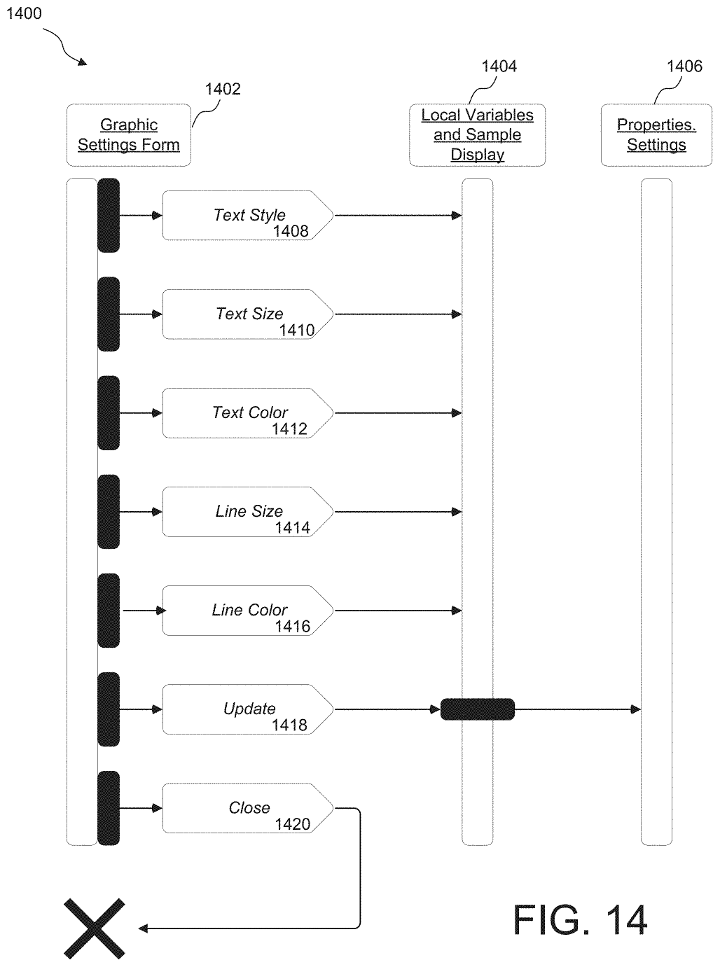

FIG. 14 is a graphical user interface (GUI) framework 1400 according to one embodiment of the disclosure. The first layer is graphic settings form 1402. The second layer is local variables and sample display 1404. The third layer is properties 1406.

The graphic settings form 1402 includes text style 1408, text size 1410, text color 1412, line size 1414, line color 1416, update 1418, and close 1420.

In the above disclosed embodiments, similar terms means similar elements. Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present invention, disclosure, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A computational architecture for managing medical images, comprising
a first server;
a medical imaging device, the medical imaging device communicating to the first server to verify the first server is online, a first image being taken by the medical imaging device is in a first format,
wherein the first server is communicable with a primary database through a firewall, the first server deposits the first image into a primary database; and
a second server, the second server being communicable with the primary database, wherein the second server transforms the first image into a second image, the second image is in a second format different from the first format.

2. The computational architecture for managing medical images according to claim 1, further comprising
a secondary database communicable with the first server through a firewall, the secondary database being communicable with the second server only when the primary database is interrupted, the secondary database not being communicable with the primary database.

3. The computational architecture for managing medical images according to claim 1, further comprising
a client browsing device, the client browsing device is at least one selected from followings: a laptop, a desktop, a mobile phone, and a tablet.

4. The computational architecture for managing medical images according to claim 1, wherein the first format is DICOM.

5. The computational architecture for managing medical images according to claim 1, wherein the second format is one selected from followings: JPG, GIF, PNG, TIFF, and BMP.

6. The computational architecture for managing medical images according to claim 1, wherein the first server encrypts the first image with a first key, the first key is a private key associated with a clinic that operates the medical imaging device.

7. The computational architecture for managing medical images according to claim 6, wherein the second server decrypts the first image with a second key, the second key is a community-of-interest key specific to a client, the first key and the second key are independent from each other.

8. The computational architecture for managing medical images according to claim 1, wherein the second server receives a request to view an image.

9. The computational architecture for managing medical images according to claim 8, wherein the second image includes data overlays on the first image.

10. The computational architecture for managing medical images according to claim 1, wherein the first server is the second server.

11. A computing architecture for managing medical images, comprising
a server, the server including at least a processor and a machine readable memory accessible by the processor, the server performing the followings:
receiving a request from an image browsing device to access a first image, the first image being in a first format;
conducting an authentication process for the image browsing device;
conducting an authorization process for the image browsing device;
accessing the first image from a primary database;
transforming the first image into a second image, the second image being in a format different from the first format; and
sending the second image to the image browsing device.

12. The computing architecture for managing medical images according to claim 11, wherein the first format is DICOM.

13. The computing architecture for managing medical images according to claim 11, wherein the second format is one selected from followings: JPG, GIF, PNG, TIFF, and BMP.

14. The computing architecture for managing medical images according to claim 11, wherein the authentication process includes verifying username, password, and biometric data.

15. The computing architecture for managing medical images according to claim 11, wherein the authorization process includes verifying a community-of-interest key and/or certificate.

16. The computing architecture for managing medical images according to claim 15, wherein the first image was encrypted before being accessed by the server, the first image was encrypted with a first key not associated with the community-of-interest key.

17. The computing architecture for managing medical images according to claim 15, wherein the first image is decrypted using the community-of-interest key before being transformed into the second image.

18. The computing architecture for managing medical images according to claim 11, wherein the second image covers only a portion of the first image.

19. The computing architecture for managing medical images according to claim 11, wherein the image browsing device is one selected from followings: a laptop, a desktop, a mobile phone, and a tablet.

20. The computing architecture for managing medical images according to claim 11, further comprising
a secondary database separate from the primary database, the secondary database stores a same copy of the first image, the secondary database is accessible by the server only when the primary database is not accessible.
